(19) United States
(12) Patent Application Publication
(10) Pub. No.: US 2025/0082547 A1
(43) Pub. Date: Mar. 13, 2025

(54) System and Method for Medication Management

(71) Applicant: Charles Moore, Orange Beach, AL (US)

(72) Inventor: Charles Moore, Orange Beach, AL (US)

(21) Appl. No.: 18/464,547

(22) Filed: Sep. 11, 2023

(51) Int. Cl. A61J 7/04; A61J 1/16; G07C 9/00; G16H 20/13

(52) U.S. Cl. CPC A61J 7/0481; A61J 1/16; A61J 7/0454; G07C 9/00896; G16H 20/13; A61J 1/03; A61J 2205/60

(57) ABSTRACT

The present invention discloses a system and method for medication management, the system comprising a smart device utilized by a first user, a medication packaging designed to store one or more medications and having a backing layer removably attachable to the smart device, one or more capsules for medication storage, and a medication tracking mechanism communicatively coupled to the smart device. The smart device interacts with the medication tracking mechanism, facilitating data exchange and real-time monitoring. Some embodiments include a user-facing screen and speakers to provide medication alerts, and the capability to send GPS coordinates and medication data of the smart device to a server device for access by other users.

### TECHNICAL FIELD

The present invention relates generally to medication management systems, and more particularly, to a system and method for managing medications including a smart device and a removably attachable medication packaging having a medication tracking mechanism.

### BACKGROUND OF THE INVENTION

Medication non-adherence is a prevalent issue that can lead to suboptimal health outcomes for patients, causing significant distress for both patients and their caregivers. This problem becomes even more critical in emergency or life-threatening situations, as well as for individuals with severe mental illness. In addressing this challenge, the concept of “digital pills” has emerged as a potential solution. These innovative pills incorporate a sensor within the medication, enabling the tracking of medication ingestion. By leveraging this technology, digital pills offer a promising avenue to tackle the issue of medication non-adherence and improve patient outcomes.

Furthermore, there is currently a wealth of companies engaged in the development of ingestible sensors, primarily in the USA and Japan. This market sector is anticipated to flourish as more companies venture into this field. The COVID-19 pandemic has heightened the significance of telemedicine, leading to expectations of substantial growth in the ingestible sensor market until at least 2027, as predicted by Market Research Future.

These new sensors are transforming the landscape of digital medicine, a rapidly evolving medical discipline that leverages digital tools to enable more personalized patient care. Some experts foresee a new wave of digital medicine that will utilize Artificial Intelligence (AI) to analyze data gathered from various sources. This could facilitate highly personalized treatment plans, such as adjusting medication dosage, and promote open communication between patients and physicians regarding these changes. However, as this technology progresses, crucial factors such as control and privacy of patient data, cost-effectiveness, and patient acceptance will remain major considerations.

Upon reviewing previous inventions in the realm of medication management, many promising solutions have been proposed. However, these technologies have often struggled to achieve widespread adoption among everyday patients. This is largely due to their high cost, escalating challenges associated with their use, and complex system requirements.

### BRIEF SUMMARY OF THE INVENTION

To address the drawbacks of earlier inventions, a new system, disclosed herein, is proposed. This system centers around unique packaging that can accommodate any type of medication and can be attached to any smart device, enhancing both portability, connectivity, quality, and safety. The assembly can monitor a patient's medication intake routine and relay this information to connected systems, ideally those operated by healthcare professionals, to improve remote patient management. Additionally, the system can assist in connecting a user, who needs a specific medication, with one or more other users within a certain geographical proximity that have access to the specific medication. This connectivity could facilitate quicker and more efficient access to necessary medications, particularly in emergency scenarios.

The system comprises a smart device with a memory and at least one processor used by a first user. Additionally, the system includes a medication packaging configured to store one or more medications. The medication packaging comprises a backing layer and one or more capsules connected to the backing layer, with each capsule configured to store a respective medication. The system further includes a medication tracking mechanism communicatively coupled to the smart device.

The memory of the smart device stores computer program instructions, and the at least one processor is configured to execute the computer program instructions and communicatively couple to the medication tracking mechanism. This enables the smart device to effectively manage medication-related information and interact with the medication tracking mechanism.

In one aspect, the backing layer of the medication packaging is removably attached to the smart device, allowing for convenient attachment and detachment of the medication packaging.

In another aspect, the backing layer is magnetic, providing a secure attachment mechanism for the medication packaging to the smart device.

The medication tracking mechanism incorporated into the medication packaging utilizes various technologies, including radio-frequency identification (RFID), near-field communication (NFC), sensors, circuits, barcodes, or a combination thereof. This enables accurate tracking and identification of medications within the medication packaging.

The smart device, when communicatively coupled with the medication tracking mechanism, is configured to receive data from and/or exchange data with the medication tracking mechanism. This facilitates real-time monitoring and synchronization of medication-related information between the smart device and the medication packaging.

The smart device further comprises a user-facing screen and/or speakers. The computer program instructions stored in the memory of the smart device are configured to cause the at least one processor to provide an alert indicating that it is time to take a medication. This enhances medication adherence by notifying the user when it is time to take their medication.

According to some possible implementations in which systems and/or methods, described herein, may be implemented, a server device having a processor, a memory, and one or more programs stored within memory for facilitating the exchange of information between two or more smart devices can be communicatively coupled to a first smart device via an installed application and to a second smart device via an installed application.

When the one or more programs stored within the server device's memory are run on the processor, the server device is configured to receive personal information from the first smart device and the second smart device.

The received personal information from both devices can be stored in the server's memory.

According to some possible implementations, the server device is a web server.

According to some possible implementations, the server device is a cloud server.

According to some possible implementations, when the server device is coupled to a smart device, the server device periodically receives GPS coordinates relating to the position of the user device.

Additionally, the computer program instructions are further configured to cause the at least one processor to send a first set of GPS coordinates relating to the physical location of the smart device and data relating to the medications actively stored in the medication packaging to a server device communicatively coupled to the smart device. The server device receives this data and generates an alert when a second user having a second smart device is within a preset distance of the first set of GPS coordinates and requires one or more medications stored in the medication packaging.

To ensure user privacy and consent, the computer program instructions prompt the first user to select whether to share their location with the second user in response to receiving the alert. Based on the first user's confirmation, the computer program instructions cause the server device to send data corresponding to the first user's confirmation and the first set of GPS coordinates to the second device.

From the applicant's perspective, it has been observed that none of the prior art methods entirely fulfill the existing needs and can be rather unwieldy. Consequently, there's a necessity for a solution in the prior art that effectively eliminates the aforementioned inconveniences, challenges, and complexities.

The present invention introduces such a solution. To the best of the applicant's knowledge, the unique combination of features offered by this invention has not been disclosed in any of the cited prior art, which seems to represent the general field of this technology. However, it's important to note that the cited prior art may not represent an exhaustive list of all relevant prior art.

### BRIEF DESCRIPTION OF THE DRAWINGS

The invention will be readily understood by the following detailed description in conjunction with the accompanying drawings, wherein like reference numerals designate like structural elements, and in which:

FIG. 1 is a perspective view of an embodiment of the disclosure.

FIG. 2 is an exploded view thereof.

FIG. 3 is a diagram of an example implementation in which systems and/or methods, described herein, may be implemented.

FIG. 4 is a flow chart of an example process for facilitating emergency medicine transfers between users of the system.

FIG. 5 is a diagram of example components of a computing device.

The drawings described herein are for illustration purposes and are not intended to limit the scope of the present subject matter in any way.

### DETAILED DESCRIPTION

In the following description, to better understand the aforementioned purposes, features, and advantages of the present invention, numerous specific details are set forth in order to provide a thorough understanding of the claimed subject matter. It should be noted that these details and examples are provided to merely aid in understanding the descriptions, and they do not, in any way, limit the scope of the present invention. The present invention can also be implemented in other modes different from those described herein and the present invention is not limited to the specific embodiments disclosed below.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The specification may refer to “an”, “one” or “some” embodiment(s) in several locations. This does not necessarily imply that each such reference is to the same embodiment(s), or that the feature only applies to a single embodiment. A single feature of different embodiments may also be combined to provide other embodiments.

Furthermore, as used herein, the singular forms “a”, an” and “the” are intended to include the plural forms as well, unless expressly stated otherwise, It will be further understood that the terms “includes”, comprises “, including” and/or “comprising” when used in this specification, specify the presence of stated features, integer steps, operations, elements and/or components, but do not preclude the presence or addition of one or more other features integers, steps, operations, elements, components, and/or groups thereof. As used herein, the term “and/or” includes any and all combinations and arrangements of one or more of the associated listed items.

Within this disclosure, the terms “user device” and “smart device” are used interchangeably.

The computer-readable storage medium (“memory”) can be a tangible device that can retain and store instructions for use by an instruction execution device. The computer-readable storage medium may be, without limitation, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including, but not limited to, an object-oriented programming language such as Python, Java, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus, or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Unless otherwise defined, all terms (including technical and scientific terms) used herein have the same meaning as commonly understood by one of ordinary skill in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### System Overview

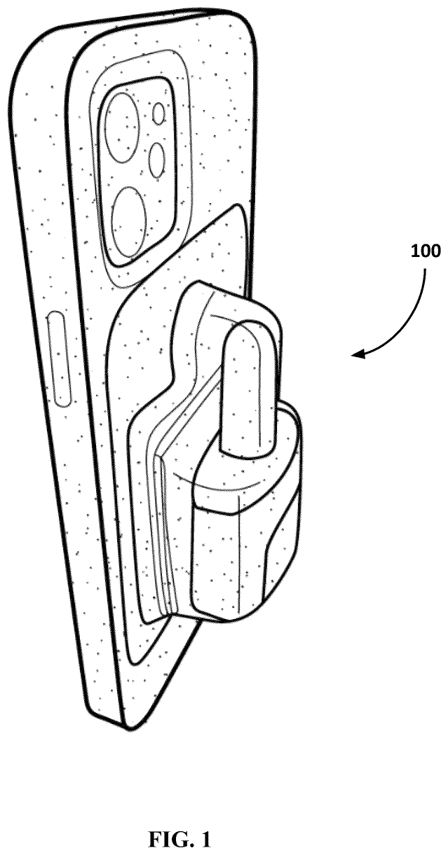

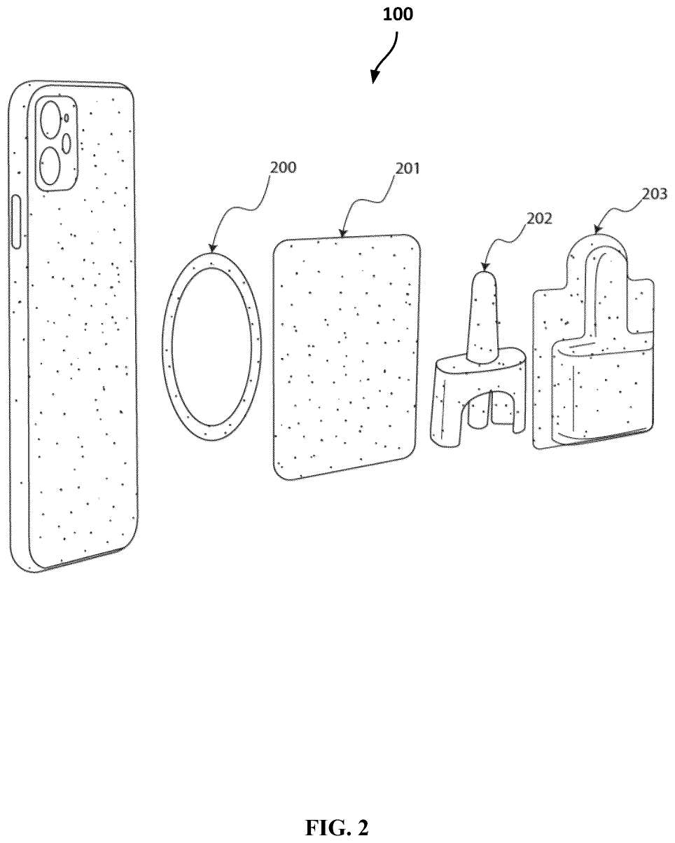

Referring now to FIG. 1 and FIG. 2, an embodiment of a system for medication management 100 according to one aspect of the disclosure is illustrated. The system for medication management comprises a smart device, a medication packing configured to store one or more medications, and a medication tracking mechanism. The smart device has a memory and at least one processor, wherein the at least one processor is configured to execute the computer program instructions stored in the memory and communicatively couple to the smart device with the medication tracking mechanism. The medication packing comprises a backing layer 200, 201 and a capsule connected to the backing layer configured to store a medication 203. The backing layer is magnetized or comprised partially of a ferrous material and configured to be removably attached to the smart device. The magnetized or ferrous metal material may be, for example, a flexible magnetic or ferrous metal material tape or a rigid magnetic or ferrous metal material plate. This material guarantees a robust and secure connection to the device, minimizing potential loss or damage. A nozzle 202 facilitates the withdrawal of medication from the capsule.

### Medication Tracking Functionality

The medication tracking mechanism is configured to be communicatively coupled to the smart device. This tracking mechanism can utilize medication labels, RFID, NFC, SansEC, sensors, circuits, barcodes, combinations thereof, or other methods for sending and/or exchanging data relating to medication names, dosage, expiration dates, batch numbers, manufacturers, drug interactions, potential side effects, storage conditions, active and inactive ingredients, the conditions or diseases the medications are intended to treat, contraindications, frequency of use, route of administration, quantity of medication remaining, prescription number, prescribing physician, date of prescription, usage history, refill information, warning, costs and insurance information, special instructions, and the patient's name with the smart device.

For embodiments having a medication tracking mechanism that utilizes medication labels or barcodes, the smart device can be configured to use video processing techniques to read the medication information from a medication label through optical character recognition and image processing algorithms.

For embodiments having a medication tracking mechanism that utilizes an RFID chip/tag, the smart device can be configured with an RFID reader or used with an external reader connected to the smart device via USB, Bluetooth, or other communication methods. Alternatively, the smart device can be equipped with NFC capabilities configured to read RFID.

For embodiments having a medication tracking mechanism that utilizes an NFC tag that stores pertinent medication data, the smart device can be configured with NFC capabilities to read this data.

For embodiments having a medication tracking mechanism that utilizes wireless, open-circuit sans electrical connections (“SansEC”), the medication packaging would feature a SansEC sensor embedded into the label or even the packaging material itself. This sensor would store data including, but not limited to, the medication's name, dosage, expiration date, and possibly even temperature or spoilage information. For such embodiments, smart devices can be configured to natively support SansEC interrogation or connect to an external device for SansEC interrogation to retrieve the medication information. From a technical perspective a SansEC sensor becomes electrically active upon interrogation and emits a wireless response. The smartphone captures this response, which includes various attributes like frequency, amplitude, and bandwidth of the magnetic field. These attributes correspond to the physical property states (e.g., medicine type, temperature, spoilage level) stored or measured by the sensor.

In the preferred embodiment, the smart device further comprises a user-facing screen and/or speakers and wherein the computer program is further configured to cause the at least one processor to provide an alert indicating that it is time to take a medication.

### System Working Environment

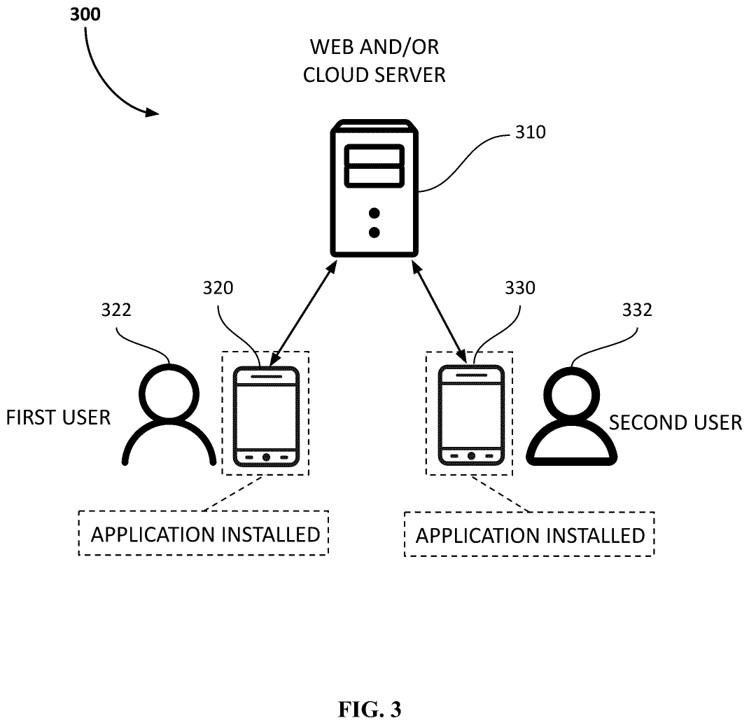

FIG. 3 is a diagram of an example implementation in which systems and/or methods, described herein, may be implemented. As shown in FIG. 3, the example environment 300 includes a first smart 322, a first smart device 320, a web/cloud server 310, a second smart device 16, and a second user 322. The first smart device is associated with the first user and the second smart device is associated with the second user. The first smart device has an application installed. Similarly, the second smart device has an application installed. These software applications include, without limitation, a set of tools for communicatively coupling and interacting with the server 310. The server 310 has a memory and one or more processors capable of running one or more programs.

When the one or more programs stored within the server device's memory are run on the processor, the server device is configured to receive personal information from the first smart device and the second smart device. The received personal information from both devices can be stored in the server's memory.

According to some possible implementations, when the server device is coupled to a smart device, the server device periodically receives GPS coordinates relating to the position of the smart device.

Additionally, the computer program instructions stored in the memory of the first smart device are further configured to cause the at least one processor to send a first set of GPS coordinates relating to the physical location of the first smart device and data relating to the medications actively stored in the medication packaging to the server device. The server device receives this data and generates an alert when a second user having a second smart device is within a preset distance of the first set of GPS coordinates and requires one or more medications stored in the medication packaging.

To ensure user privacy and consent, the computer program instructions prompt the first user to select whether to share their location with the second user in response to receiving the alert. Based on the first user's confirmation, the computer program instructions cause the server device to send data corresponding to the first user's confirmation and the first set of GPS coordinates to the second device.

Computing devices of the embodiment 300 may interconnect via wired connections, wireless connections, or a combination of wired and wireless connections. In some possible implementations, the computing devices may interconnect with a cellular network (e.g., a long-term evolution (LTE) network, a code division multiple access (CDMA) network, a 3G network, a 4G network, a 5G network, another type of next generation network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, a cloud computing network, and/or the like, and/or a combination of these or other types of networks.

The number and arrangement of devices shown in FIG. 3 are provided as an example. In practice, there may be additional devices and/or networks, fewer devices and/or networks, different devices and/or networks, or differently arranged devices and/or networks than those shown in FIG. 3. Furthermore, two or more devices shown in FIG. 3 may be implemented within a single device, or a single device may be implemented as multiple, distributed devices. Additionally, or alternatively, a set of devices (e.g., one or more devices) of the aforementioned environments 300 may perform one or more functions described as being performed by another set of devices of the environment.

### Emergency Medicine Transfer Process Between Two Users

Methods disclosed herein revolve around a community-centric approach to medication management, harnessing the interconnectedness of smart devices. These methods capitalize on a unique combination of location services and a centralized communication system, resulting in a robust support mechanism for individuals seeking specific medications.

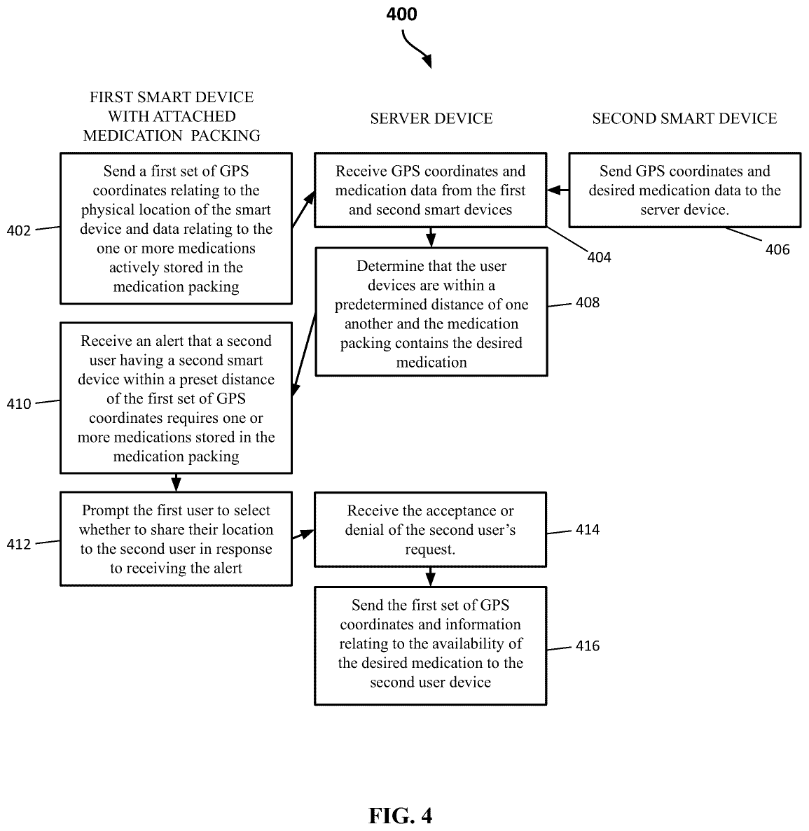

FIG. 4 illustrates an example process 400 for facilitating emergency medicine transfers between users of the system. At step 402, a first smart device with an attached medication packing sends a first set of GPS coordinates relating to the physical location of the device and data relating to the one or more medications actively stored in the medication packing to the server device. In practice, this information is periodically sent to the server while the first smart device is communicatively coupled to the server device.

If a second user requires a desired medication in an emergency scenario and elects to use the disclosed system to attempt to obtain the desired medication, the second user can use the application stored on the second smart device to send, at step 406, GPS coordinates relating to the position of the second smart device and desired medication data to the server.

At step 408, once the server device receives the GPS coordinates and medication data from the first and second smart devices 404, the server device then determines that the user devices are within a predetermined distance of one another and the medication packing of the first smart device contains the desired medication. If these conditions are met, at step 410 the server device sends an alert to the first smart device that a second smart device within a preset distance of the first smart device requires one or more medications stored in the first smart device's medication packing. Accordingly, the first user is prompted to select whether to share their location and information regarding the availability of the desired medication with the second user 412. The first user's acceptance or denial is sent to the server.

In response to receiving the acceptance of the second user's request by the first user, the server sends the first set of GPS coordinates and information relating to the availability of the desired medication to the second user device. In some embodiments, this information further includes information relating to methods of contacting the first user, such as their phone number. In some embodiments, the applications stored on the first and second smart devices allow the first user and second user to track each other's locations and/or message one another in live time in order to facilitate their meeting and the transfer of the desired medication.

FIG. 4 outlines a process 400 to facilitate emergency medicine exchanges between system users. At step 402, the first smart device, having an attached medication package, shares its current GPS location and information about the medicines it contains with the server. Regularly, this information is sent to the server as long as the first smart device remains connected.

If there's a second user in urgent need of medication, and they choose to utilize the system for this purpose, they can activate an app on their second smart device. At step 406, the second smart device sends its GPS location and details of the desired medication to the server.

By step 408, once the server receives data from both the first and second smart devices 404, it checks if: a) they're in proximity to each other and b) the medication package of the first smart device contains the required medication. If both these criteria are satisfied, at step 410 the server sends a notification to the first smart device indicating that a nearby second smart device is in need of its stored medications. Subsequently, the first user gets a prompt 412 asking if they're willing to share their location and medication details with the second user. The server then gets notified of their choice.

If the first user agrees to the request of the second user, the server provides the second smart device with the location of the first smart device and details about the available medication 416. Some variations of this system might also share the first user's contact methods, like a phone number, with the second user. Furthermore, the apps on both the first and second smart devices might have features allowing the two users to track each other in real-time or chat directly, making their meetup and the medication transfer more efficient.

### Emergency Medicine Request Broadcast

In another scenario, when the first user is in need of one or more medications, additional program instructions prompt the device's internal processor to undertake two pivotal actions:

The first action is to ascertain and dispatch the device's current geographical coordinates, obtained from its GPS system, to an external server device. This ensures that the overarching system remains privy to the user's real-time location.

Concurrently, the smart device forwards specific data regarding the medications the user is actively in pursuit of to the same server device. This information might range from the exact medication names to doses or other pertinent specifications.

Upon receipt of this twofold data—the geographical location and medication particulars—the server device broadcasts a comprehensive alert to a network of other smart devices that are communicatively coupled to it. This alert comprises a package of critical information that encapsulates both the originating user's location and the medication details, allowing other users the opportunity to assist the first user.

### Unused Medication Functionality

The system disclosed herein can be utilized to address the significant issue of unused medications which represent a costly dilemma, both financially and environmentally. Unused medications can lead to several complications such as drug addiction, threats to children, environmental concerns, and improper medication use. Notably, a vast number of people cannot access medications due to cost, yet a plethora of medications remain unused.

With the majority of unused medication initiatives being “Take-Back” programs, some embodiments of the disclosed system can play a pivotal role. Such embodiments offer alerts to users about available Take-Back options, guide them with directions to drop-off locations, and incentivize them with rewards like coupons or credits for proper medication return. Moreover, in scenarios where Take-Back isn't an option, the system can help users identify if their medication is safely flushable. The platform could even pave the way for novel Take-Back strategies like additional collection points or home pick-up services.

Medication re-use, though less common due to concerns about quality and safety, finds its application in settings like senior living communities or nursing homes. Here, the system platform's technology ensures the quality of unused medications by providing critical information about them. For instance, the system can inform about expiration dates and other details about previously distributed medications, ensuring their safe re-use.

Take for example the user of the disclosed system with traditional medication blister packs. In this setup, each tablet is stored in a distinct blister, and the platform can offer data about the unused part of the medication. Future innovations might encompass smart pill dispensing containers that allow access to only one dosage at a time while safely securing the rest. This ensures that any returned medications come with comprehensive information about the remaining medications' quality and potency.

Finally, some embodiments of the disclosed system can be configured to reward users for utilizing safe medication destruction systems. For example, if a user of the disclosed system elects to not take the remainder of the medication stored in the medication packaging, they can remove the medication, insert the medication in a Deterra® Drug Deactivation and Disposal Pouch or the like, seal the pouch, and submit a picture of the sealed pouch to a server associated with tracking user data and medication compliance within a given time frame of the medication tracking system detecting that the medication has been removed from the medication packaging and properly disposed of. Confirmation of proper disposal of the medication can be associated with a monetary benefit, rebate, or other reward for the user. Alternate methods of verifying that the medication has been properly disposed of known in the art can be used as well.

### Underlying Technology

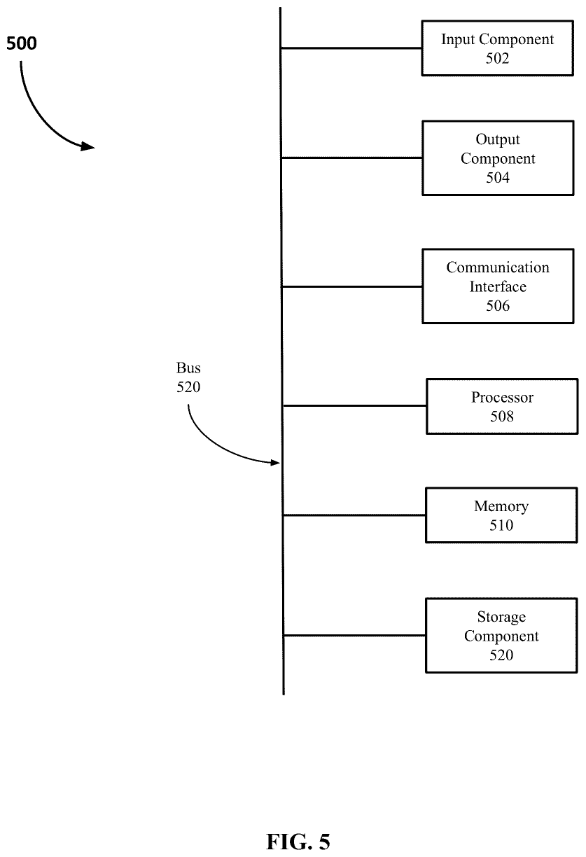

FIG. 5 is a diagram of example components of a computing device 500. Computing device 500 may correspond to first smart device 320, second smart device 330, and/or web/cloud server 310. In some implementations, the smart devices and/or server device may include one or more devices 500 and/or one or more components of device 500. As shown in FIG. 5, device 500 may include an input component 502, an output component 504, a communication interface 506, a processor 508, a memory 510, a storage component 512, and a bus 514.

The bus 514 includes a component that permits communication among the components of device 500. Processor 508 is implemented in hardware, firmware, or a combination of hardware and software. Processor 508 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 508 includes one or more processors capable of being programmed to perform a function. Memory 510 includes a random access memory (RAM), a read only memory (ROM), and/or another type of dynamic or static storage device (e.g., a flash memory, a magnetic memory, and/or an optical memory) that stores information and/or instructions for use by processor 508.

Storage component 512 stores information and/or software related to the operation and use of device 500. For example, storage component 512 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

Input component 502 includes a component that permits device 500 to receive information, such as via user input (e.g., a touch screen display, a keyboard, a keypad, a mouse, a button, a switch, and/or a microphone). Additionally, or alternatively, input component 502 may include a sensor for sensing information (e.g., a global positioning system (GPS) component, an accelerometer, a gyroscope, and/or an actuator). Output component 504 includes a component that provides output information from device 500 (e.g., a display, a speaker, and/or one or more light-emitting diodes (LEDs)).

Communication interface 506 includes a transceiver-like component (e.g., a transceiver and/or a separate receiver and transmitter) that enables device 500 to communicate with other devices, such as via a wired connection, a wireless connection, or a combination of wired and wireless connections. Communication interface 506 may permit device 500 to receive information from another device and/or provide information to another device. For example, communication interface 506 may include an Ethernet interface, an optical interface, a coaxial interface, an infrared interface, a radio frequency (RF) interface, a universal serial bus (USB) interface, a wireless local area network interface, a cellular network interface, and/or the like.

Device 500 may perform one or more processes described herein. Device 500 may perform these processes based on processor 508 executing software instructions stored by a non-transitory computer-readable medium, such as memory 510 and/or storage component 512. A computer-readable medium is defined herein as a non-transitory memory device. A memory device includes memory space within a single physical storage device or memory space spread across multiple physical storage devices.

Software instructions may be read into memory 510 and/or storage component 512 from another computer-readable medium or from another device via communication interface 506. When executed, software instructions stored in memory 510 and/or storage component 512 may cause processor 508 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of components shown in FIG. 5 are provided as an example. In practice, device 500 may include additional components, fewer components, different components, or differently arranged components than those shown in FIG. 5. Additionally, or alternatively, a set of components (e.g., one or more components) of device 500 may perform one or more functions described as being performed by another set of components of device 500.

A second embodiment of a system for medication management further includes a locking mechanism for the capsules in the medication packaging. In this embodiment, the capsules are selectively lockable to the backing layer and can only be unlocked upon receipt of an authorization signal from the smart device. This introduces an additional layer of security, ensuring that the medication can only be accessed under specific conditions determined by the smart device, thus reducing the risk of unauthorized or accidental medication intake.

In this embodiment, the smart device generates an authorization signal based on a variety of conditions. These can include the user entering an authorization code via a specialized app on the smart device, verification of the user's prescription through an online database, or the physical connection of the smart device with the medication packaging. Additionally, the signal can be triggered when the smart device determines it's the appropriate time for the user to take a specific medication stored in a particular capsule. These authorization methods can be used individually or in combination, providing multiple layers of security before unlocking access to the medication. This enhances the existing locking mechanism for the capsules, further ensuring the system's goal to reduce unauthorized or accidental access to medication.

This embodiment expands on the capabilities of the smart device's processor and stored computer program instructions to include more advanced decision-making functionalities. In this version, the smart device not only provides an alert indicating that it's time to take a medication but also determines the “appropriate time period” for the user to take a specific medication stored in a particular capsule. In some embodiments, these determinations can be made based on data obtained by the smart device from one or more components of the medication packaging.

In a third embodiment of a system for medication management, the smart device serves as the central controller, ensuring both the secure and accurate dispensing and mixing of medications. Operated by a first user, the smart device contains a memory for storing computer program instructions and at least one processor for executing these instructions. The device pairs with the medication packaging through various means of communication, such as radio-frequency identification (RFID), near-field communication (NFC), sensors, circuits, or barcodes. This enables the medication tracking mechanism, which could comprise one or more of these technologies, to provide real-time information about the types, quantities, and authorized combinations of medications or medication ingredients stored in individual capsules.

Upon receiving either a user-authorized request or instructions from a healthcare provider, the smart device sends an initial authorization signal to unlock specific capsules. The capsules' locking mechanisms are engineered to only disable upon receiving this proper authorization from the smart device. Once unlocked, the capsules release their contents into a medication mixing chamber. In some embodiments, this chamber is not only selectively lockable but also configured to allow the mixing of medications and/or medication ingredients from different capsules, but only after receiving a mixing authorization signal from the smart device. This ensures that only authorized combinations are mixed together.

After the mixing is completed to satisfaction, a secondary signal is sent from the smart device to confirm that the medications have been properly mixed. Upon receiving this second unlocking command, the locking mechanism of the mixing chamber is disabled, making the mixed medication unlockable or removable from the chamber. This enables the user or healthcare provider to safely administer the precisely mixed dose, ensuring both safety and efficacy in medication management.

In a fourth embodiment of a system for medication management, the smart device includes a remote prescription and authorization module. This module serves multiple functions:

As needed, it enables a consultation between the first user and a healthcare provider through a communication interface on the smart device. This consultation can lead to the issuance of an initial prescription for one or more medications.

The module can authenticate this initial prescription by establishing a communicative link between the medication packaging and the smart device. This authentication allows for the dispensing of the prescribed medication.

The module can also communicate with various dispensing entities to release the medication. These entities could be a remote pharmacy kiosk, a mobile dispensing van, a fixed dispensing machine, or another individual authorized to dispense medication and located near the first user.

For added security, the module enables an additional layer of authentication. The medication packaging can either be physically attached to the smart device or communicate wirelessly with it to confirm that the authorized medication has been dispensed.

The foregoing disclosure provides illustration and description, but is not intended to be exhaustive or to limit the implementations to the precise form disclosed. Modifications and variations are possible in light of the above disclosure or may be acquired from practice of the implementations.

It will be apparent that systems and/or methods, described herein, may be implemented in different forms of hardware, firmware, or a combination of hardware and software. The actual specialized control hardware or software code used to implement these systems and/or methods is not limited to the implementations. Thus, the operation and behavior of the systems and/or methods were described herein without reference to specific software code-it being understood that software and hardware may be designed to implement the systems and/or methods based on the description herein.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

I claim:

1. A system for medication management, the system comprising:
a smart device having a memory and at least one processor used by a first user;
a medication packaging configured to store one or more medications, the medication packaging comprising:
a backing layer;
one or more capsules connected to the backing layer, each of the capsules configured to store a respective medication, and,
a medication tracking mechanism configured to be communicatively coupled to the smart device;
wherein the memory stores computer program instructions; and,
wherein the at least one processor is configured to execute the computer program instructions and communicatively couple to the medication tracking mechanism.

2. The system of claim 1 wherein the backing layer is configured to be removably attached to the smart device.

3. The system of claim 2, wherein the backing layer is magnetic.

4. The system of claim 3, wherein the medication tracking mechanism comprises radio-frequency identification, near-field communication, sans electrical connections, sensors, circuits, barcodes, or a combination thereof.

5. The system of claim 4, wherein the smart device, when communicatively coupled with the medication management device, is configured to receive data from and/or exchange data with from the medication tracking mechanism.

6. The system of claim 5, wherein the smart device further comprises a user-facing screen and/or speakers and wherein the computer program is further configured to cause the at least one processor to provide an alert indicating that it is time to take a medication.

7. The system of claim 4, wherein the computer program is further configured to cause the at least one processor to:
send, to a server device communicatively coupled to the smart device, a first set of GPS coordinates relating to the physical location of the smart device and data relating to the one or more medications actively stored in the medication packaging;
receive, by a server device, an alert that a second user having a second smart device within a preset distance of the first set of GPS coordinates requires one or more medications stored in the medication packaging;
prompt the first user to select whether to share their location to the second user in response to receiving the alert; and,
send, by a server device and to the second device, data corresponding to the first user's confirmation and the first set of GPS coordinates.

8. The system of claim 4, wherein:
the computer program instructions stored in the memory of the smart device further cause the at least one processor to:
transmit, to a server device, a first set of GPS coordinates representing the physical location of the smart device; and
send data relating to the one or more medications actively sought by the user of the smart device;
the server device, upon receiving the GPS coordinates and the medication data:
is configured to broadcast an alert to a plurality of other smart devices communicatively coupled thereto, such alert including the received location and medication data;
the purpose of said broadcast being to facilitate the assistance of the first user by users of the other smart devices.

9. The system of claim 2, wherein the smart device further comprises a remote prescription and authorization module configured to:
enable a consultation between the first user and a healthcare provider via a communication interface on the smart device, wherein the consultation results in the issuance of an initial prescription for one or more medications; and/or
authenticate the initial prescription through a communicative link established between the medication packaging and the smart device, wherein said authentication serves to authorize the dispensing of the prescribed medication; and/or
communicate with a dispensing entity selected from the group consisting of a remote pharmacy kiosk, a mobile dispensing van, a fixed dispensing machine, and another individual authorized to dispense medication in proximity to the first user for the purpose of dispensing one or more dosages of the medication; and/or
enable an additional layer of authentication by allowing the medication packaging to be either physically attached to the smart device or wirelessly communicating with the smart device to confirm the dispensing of the authorized medication.

10. A system for medication management, the system comprising:
a smart device having a memory and at least one processor operated by a first user;
a medication packaging configured to store one or more medications and further configured to be communicatively coupled to the smart device, the medication packaging comprising:
a backing layer;
one or more capsules selectively lockable to the backing layer, each of the capsules configured to store a respective medication and being authorized for unlocking only upon receipt of an authorization signal from the smart device;
a medication tracking mechanism;
wherein the memory of the smart device stores computer program instructions;
wherein the locking mechanism of the one or more capsules is configured to be disabled only upon receiving an unlocking command that confirms proper authorization from the smart device; and,
wherein the at least one processor is configured to execute the stored computer program instructions.

11. The system of claim 10, wherein the backing layer is configured to be removably attached to the smart device.

12. The system of claim 11, wherein the attachment of the smart device to the backing layer triggers the at least one processor to send an authorization signal to the medication packaging.

13. The system of claim 12, wherein the backing layer comprises magnetic properties.

14. The system of claim 13, wherein the medication tracking mechanism comprises one or more of the following: radio-frequency identification, near-field communication, sans electrical connections, sensors, circuits, and barcodes.

15. The system of claim 13, wherein when the smart device is communicatively coupled to the medication packaging, the smart device is configured to either receive data from the medication tracking mechanism or exchange data with the medication tracking mechanism, or both.

16. The system of claim 12, wherein:
the smart device further comprises a user-facing screen and/or speakers;
wherein the computer program instructions stored in the memory are further configured to cause the at least one processor to provide an alert indicating that it is time to take a medication; and,
wherein the authorization signal to unlock a capsule is sent only if the smart device determines that it is the appropriate time period for the user to take the specific medication stored in the capsule.

17. A system for controlled medication management and mixing, the system comprising:
a smart device having a memory and at least one processor operated by a first user;
a medication packaging configured to store one or more medications and further configured to be communicatively coupled to the smart device, the medication packaging comprising:
a backing layer;
a plurality of capsules selectively lockable to the backing layer, each of the capsules configured to store a respective medication and being authorized for unlocking only upon receipt of an authorization signal from the smart device;
a medication mixing chamber operatively connected to the capsules, configured to receive medications and/or medication ingredients from the unlocked capsules and to facilitate the mixing thereof;
a medication tracking mechanism;
wherein the memory of the smart device stores computer program instructions;
wherein the locking mechanism of the one or more capsules is configured to be disabled only upon receiving an unlocking command that confirms proper authorization from the smart device;
wherein the at least one processor is configured to execute the stored computer program instructions; and,
wherein the mixing chamber is configured to allow individual medications and/or medication ingredients from different capsules to mix together only upon receiving a mixing authorization signal from the smart device.

18. The system of claim 17, wherein:
the medication tracking mechanism comprises one or more of the following: radio-frequency identification, near-field communication, sans electrical connections, sensors, circuits, and barcodes; and,
the mixing chamber is selectively lockable and the locking mechanism of the mixing chamber is configured to be disabled only upon receiving a second unlocking command that confirms completed mixing from the smart device.

19. The system of claim 18, wherein the backing layer is configured to be removably attached to the smart device and the backing layer comprises magnetic properties.

20. The system of claim 19, wherein the attachment of the smart device to the backing layer triggers the at least one processor to send an authorization signal to the medication packaging.
